(19) United States
(12) Patent Application Publication
(10) Pub. No.: US 2026/0198784 A1
(43) Pub. Date: Jul. 16, 2026

(54) INFRARED SENSOR-BASED NANOMAGNETIC PARTICLE MEDICAL IMAGING DEVICE USING EXOTHERMIC REACTION OF PARAMAGNETIC IRON OXIDE PARTICLES, AND OPERATION METHOD THEREOF

(71) Applicant: Electronics and Telecommunications Research Institute, Daejeon (KR)

(72) Inventors: Hyo Bong HONG, Daejeon (KR); Jae Chan JEONG, Daejeon (KR); Hyeon Sung CHO, Daejeon (KR)

(21) Appl. No.: 19/312,089

(22) Filed: Aug. 27, 2025

(30) Foreign Application Priority Data
Jan. 16, 2025 (KR) 10-2025-0006969

(51) Int. Cl. A61B 5/00; A61B 5/01

(52) U.S. Cl. CPC A61B 5/0093; A61B 5/015; A61B 2560/0242

(57) ABSTRACT

The present disclosure relates to an infrared sensor-based nanomagnetic particle medical imaging device, which includes a coil configured to generate a magnetic field inside a chamber in which a measurement target is accommodated, an infrared sensor which moves along a lane installed in the chamber to detect infrared rays generated from the measurement target and nanomagnetic particles generated by the magnetic field, a temperature sensor configured to measure temperatures of the coil and the measurement target, and a processor configured to control a current applied to the coil to generate the magnetic field in the chamber, analyze data measured through the infrared sensor, and calculate heating positions of the measurement target and the nanomagnetic particles. Thus, a medical imaging device in which a heat generation function and a magnetic particle imaging (MPI) device are coupled can be formed to effectively analyze lesions.

### CROSS-REFERENCE TO RELATED APPLICATION

This application claims priority to and the benefit of Korean Patent Application No. 10-2025-0006969, filed on Jan. 16, 2025, the disclosure of which is incorporated herein by reference in its entirety.

### BACKGROUND

### 1. Field of the Invention

The present invention relates to a medical imaging device for detecting an exothermic reaction of paramagnetic iron oxide particles through an infrared sensor, and an operation method thereof.

### 2. Discussion of Related Art

Super paramagnetic iron oxide (SPIO) particles are widely used in biosensors, medical imaging, drug delivery, thermotherapy, and the like.

Position of nanomagnetic particles (SPIO) may be designated in a three-dimensional space using an AC magnetic field and a field-free line (FFL) (or a field-free point) and thus may be used in medical imaging using positioning designation characteristics. A magnetic particle imaging (MPI) device is a medical imaging device that uses these characteristics of nanomagnetic particles.

In addition, nanomagnetic particles (SPIO) generate heat only in an AC magnetic field at a specific frequency, and such a phenomenon is referred as a hyperthermia characteristic of nanomagnetic particles (SPIO).

Based on such a phenomenon, much research is being conducted on nanomagnetic particles (SPIO), and in some countries, nanomagnetic particles (SPIO) are being applied clinically on a trial basis.

In particular, MPI devices are being developed with a focus on killing cancer cells by generating hyperthermia after binding magnetic nanoparticles (SPIO) with portions near cancer tissue based on the theory that cancer tissue is weak to heat.

However, although research that combines two characteristics to simultaneously perform diagnosis and treatment is receiving much attention, there is a problem that it is difficult to distinguish the position and degree of heat generation of nanomagnetic particles.

In addition, MPI devices have a problem that a huge amount of power is consumed to generate an actual FFL and then move the actual FFL three-dimensionally, and a lot of cooling equipment is used to reduce heat generation.

To solve these problems, research is actively being conducted to apply artificial intelligence learning technologies to the research of MPI devices using nanomagnetic particles (SPIO). However, a large amount of medical data including various cases is required for artificial intelligence learning, but medical data cannot be shared for reasons of privacy and security, or only some data is allowed under limited conditions, which limits artificial intelligence learning.

The background technology of the present invention is disclosed in Korean Patent No. 10-2545062 (Jun. 14, 2023).

### SUMMARY OF THE INVENTION

The present invention is directed to providing an infrared sensor-based nanomagnetic particle medical imaging device using an exothermic reaction of paramagnetic iron oxide particles, and an operation method thereof in which whether nanomagnetic particles (super paramagnetic iron oxide (SPIO)) are bound to a desired lesion part is checked, and whether the nanomagnetic particles (SPIO) are used for generating heat and raise a temperature of a lesion by reaching a desired temperature is checked in real time.

According to an aspect of the present invention, there is provided an infrared sensor-based nanomagnetic particle medical imaging device including a coil configured to generate a magnetic field inside a chamber in which a measurement target is accommodated, an infrared sensor which moves along a lane installed in the chamber to detect infrared rays generated from the measurement target and nanomagnetic particles generated by the magnetic field, a temperature sensor configured to measure temperatures of the coil and the measurement target, and a processor configured to control a current applied to the coil to generate the magnetic field in the chamber, analyze data measured through the infrared sensor, and calculate heating positions of the measurement target and the nanomagnetic particles.

The infrared sensor-based nanomagnetic particle medical imaging device may further include a pump configured to inject a coolant into the chamber, and the processor may control the pump and the current according to the temperature of the coil measured by the temperature sensor such that the coil maintains a constant temperature.

The infrared sensor-based nanomagnetic particle medical imaging device may further include a cooling fan configured to supply cold air to the measurement target, and the processor may control the cooling fan to be driven in response to the temperature of the measurement target such that the measurement target maintains a constant temperature.

The infrared sensor may move at a predetermine speed along the lane.

The lane may include first to fourth lanes, and the infrared sensor may be provided as one or more infrared sensors disposed on each of the first to fourth lanes.

The processor may obtain information on at least three planes in consideration of a penetration depth of the infrared rays based on data of the infrared sensor measured on the first to fourth lanes.

The processor may calculate the heating position of the nanomagnetic particles based on position information obtained from at least three infrared sensors among the infrared sensors installed on the first to fourth lanes.

The processor may generate three-dimensional position information based on position information obtained from at least three infrared sensors among the infrared sensors installed on the first to fourth lanes.

The processor may generate a two-dimensional image or a three-dimensional image of the measurement target and the nanomagnetic particles.

According to another aspect of the present invention, there is provided an operation method of an infrared sensor-based nanomagnetic particle medical imaging device including applying, by a processor, a current to a coil and generating a magnetic field inside a chamber in which a measurement target is accommodated, receiving, by the processor, data about the measurement target and nanomagnetic particles measured through an infrared sensor, and analyzing, by the processor, the data measured through the infrared sensor and calculating heating positions of the measurement target and the nanomagnetic particles.

The generating of the magnetic field may include receiving, by the processor, a temperature of the coil from a temperature sensor, and controlling, by the processor, the current applied to the coil or a coolant injection according to the temperature of the coil measured through the temperature sensor.

In the generating of the magnetic field, the processor may control the coil to maintain a constant temperature.

The generating of the magnetic field may include receiving, by the processor, a temperature of the measurement target from a temperature sensor, and controlling, by the processor, a cooling fan configured to supply cold air to the measurement target.

The receiving of the data may include controlling, by the processor, the infrared sensor to move at a predetermined speed along a lane installed in the chamber.

In the receiving of the data, the lane may include first to fourth lanes, and the infrared sensor may be provided as one or more infrared sensors disposed on each of the first to fourth lanes to detect the measurement target and the nanomagnetic particles.

In the receiving of the data, the processor may obtain information on at least three planes in consideration of a penetration depth of infrared rays based on data of the infrared sensor measured on the first to fourth lanes.

In the calculating of the heating positions of the measurement target and the nanomagnetic particles, the processor may calculate the heating position of the nanomagnetic particles based on position information obtained from at least three infrared sensors among the infrared sensors installed on the first to fourth lanes.

In the calculating of the heating positions of the measurement target and the nanomagnetic particles, the processor may generate three-dimensional position information based on position information obtained from at least three infrared sensors among the infrared sensors installed on the first to fourth lanes.

In the calculating of the heating positions of the measurement target and the nanomagnetic particles, the processor may generate a two-dimensional image or a three-dimensional image of the measurement target and the nanomagnetic particles.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view schematically illustrating a configuration of an infrared sensor-based nanomagnetic particle medical imaging device using an exothermic reaction of paramagnetic iron oxide particles according to one embodiment of the present invention;

FIG. 2 is a block diagram schematically illustrating a control configuration of a medical imaging device according to one embodiment of the present invention;

FIGS. 3A and 3B are diagrams illustrating a structure for moving an infrared sensor of the medical imaging device according to one embodiment of the present invention;

FIG. 4 is an exemplary diagram illustrating a method of designating a heating position of nanomagnetic particles in the medical imaging device according to one embodiment of the present invention; and

FIG. 5 is a flowchart illustrating an operation method of a medical imaging device according to one embodiment of the present invention.

### DETAILED DESCRIPTION OF EXEMPLARY EMBODIMENTS

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, embodiments that include other components in addition to the components described in the various embodiments are also included in the scope of the present disclosure.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, when a component is referred to as being “linked,” “coupled,” or “connected” to another component, it is understood that not only a direct connection relationship but also an indirect connection relationship through an intermediate component may also be included. In addition, when a component is referred to as “comprising” or “having” another component, it may mean further inclusion of another component not the exclusion thereof, unless explicitly described to the contrary.

In the present disclosure, the terms first, second, etc. are used only for the purpose of distinguishing one component from another, and do not limit the order or importance of components, etc., unless specifically stated otherwise. Thus, within the scope of this disclosure, a first component in one exemplary embodiment may be referred to as a second component in another embodiment, and similarly a second component in one exemplary embodiment may be referred to as a first component.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, exemplary embodiments that include other components in addition to the components described in the various embodiments are also included in the scope of the present disclosure.

Hereinafter, embodiments of an infrared sensor-based nanomagnetic particle medical imaging device using an exothermic reaction of paramagnetic iron oxide particles, and an operation method thereof according to the present invention will be described.

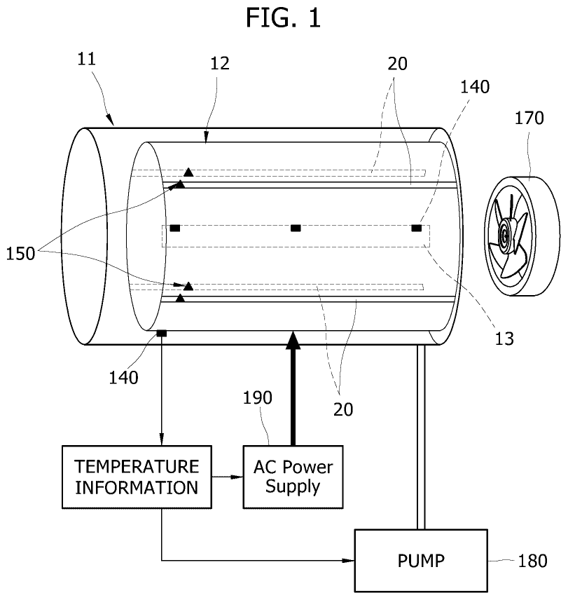

FIG. 1 is a view schematically illustrating a configuration of an infrared sensor-based nanomagnetic particle medical imaging device using an exothermic reaction of paramagnetic iron oxide particles according to one embodiment of the present invention.

Referring to FIG. 1, FIG. 1 according to one embodiment of the present invention is a view schematically illustrating the configuration of the infrared sensor-based nanomagnetic particle medical imaging device 100 using an exothermic reaction of paramagnetic iron oxide particles (hereinafter referred to as medical imaging device) according to one embodiment of the present invention.

The medical imaging device 100 includes a chamber 11 for coil temperature control, and a coil 12 for generating an AC magnetic field is provided inside the chamber. A measurement target 13 may be disposed inside the coil 12.

The medical imaging device 100 may include a plurality of infrared sensors 150 installed in the coil 12 and a temperature sensor 140 that measures a temperature of the coil 12. In addition, the medical imaging device 100 may include a power supply (AC power Supply) 190, a pump 180, and a cooling fan 170.

The medical imaging device 100 applies a current with a predetermined magnitude to the coil 12 through the power supply 190 and controls an amount of current applied to the coil 12 in response to a temperature measured through the temperature sensor 140.

In addition, the medical imaging device 100 may drive the pump 180 to supply a coolant to the coil 12 and drive the cooling fan 170 to supply cold air to the coil 12. In this case, the cooling fan 170 may operate according to a temperature of the measurement target 13 to supply cold air.

In this case, the coil 12 may perform impedance matching such that power is used to form a magnetic field without being emitted as heat. A solenoid coil may be used as the coil 12. The coil 12 generates a magnetic field according to an amount of current applied from the power supply 190. In this case, the power supply 190 may adjust an amount of current in response to a surface temperature of the coil 12.

When power is applied to the coil 12, the chamber 11 adjusts the heat generation of the coil 12 to control the coil 12 to maintain a constant temperature.

The chamber 11 may accommodate a coolant therein. The coolant may be supplied as an antifreeze coolant by the pump 180 and used to reduce the heat generation of the coil 12. The pump 180 may supply the coolant based on a surface temperature of the coil 12 measured through the temperature sensor 140.

In addition, the cooling fan 170 may be installed on a rear surface of the chamber 11. The cooling fan 170 may supply cold air to the inside of the chamber 11 according to a temperature of the temperature sensor 140 installed on the measurement target 13 inside the chamber 11. The cooling fan 170 may supply cold air to adjust a temperature of the measurement target 13.

In this case, the medical imaging device 100 obtains information about the measurement target 13 using the infrared sensor 150, and thus it is necessary to adjust a temperature of the measurement target 13 as well as a temperature of the coil 12.

The infrared sensor 150 may obtain data about the measurement target 13 while moving along a lane 20 installed in the chamber 11. Since the infrared sensor 150 uses the heat generation characteristics of nanomagnetic particles (superparamagnetic iron oxide (SPIO)), a two-dimensional or three-dimensional thermal image may be obtained through the infrared sensor.

Accordingly, the medical imaging device 100 may maintain a temperature of the coil 12 within a predetermined error range and effectively obtain data about the measurement target 13 by allowing the coil 12 to generate a magnetic field in the chamber 11.

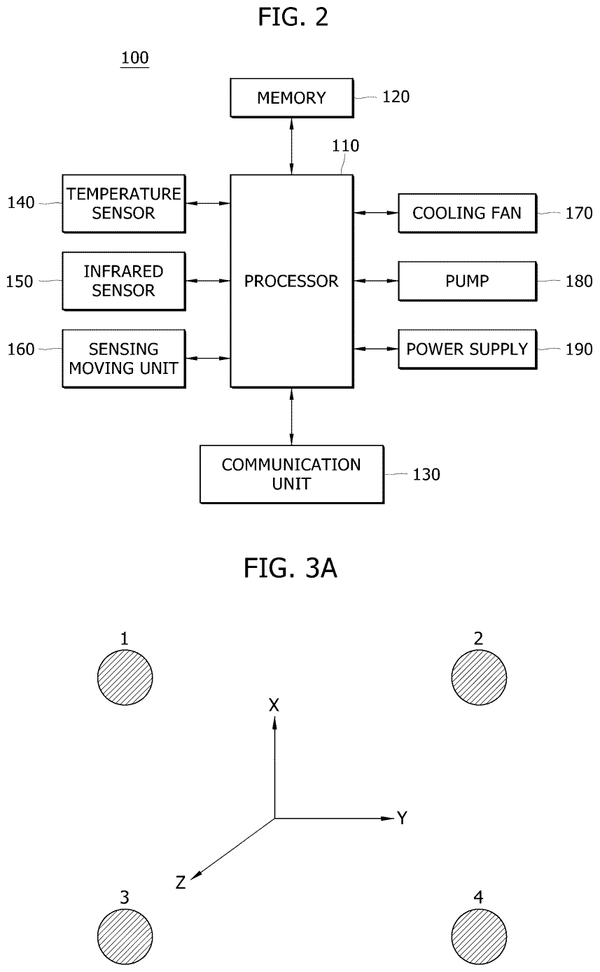

FIG. 2 is a block diagram schematically illustrating a control configuration of the medical imaging device according to one embodiment of the present invention.

Referring to FIG. 2, the medical imaging device 100 may include a memory 120, a communication unit 130, the temperature sensor 140, the infrared sensor 150, a sensor moving unit 160, the cooling fan 170, the pump 180, the power supply 190, and a processor 110.

The memory 120 may store data obtained from a plurality of temperature sensors 140 and the plurality of infrared sensors 150. The memory 120 may store control data and setting data for the pump 180, the cooling fan 170, and power supply. In addition, the memory 120 may store data about the measurement target 13 and two-dimensional or three-dimensional imaging data about the measurement target 13.

In addition, the memory 120 may store data about at least one of a data analysis algorithm, an infrared sensor position calculation algorithm, a current calculation algorithm according to a temperature, a coolant control algorithm, a fan control algorithm, a pump control algorithm, and a temperature control algorithm.

The memory 120 may include a storage device such as a non-volatile memory such as a random access memory (RAM), a read-only memory (ROM), or an electrically erased programmable ROM (EEPROM), a flash memory, a hard disk drive (HDD), or a solid state drive (SSD).

The communication unit 130 may receive data measured through the temperature sensor 140 and the infrared sensor 150, store the data in the memory 120, and transmit a control instruction of the processor 110 to a target.

The communication unit 130 may transmit or receive data between the medical imaging device 100 and another external server or terminal. In addition, the communication unit 130 may transmit or receive data through wired or wireless communication. The communication unit 130 may perform communication through at least one of short-range communication such as Ethernet, WIFI, or Bluetooth, mobile communication, and serial communication.

The plurality of temperature sensors 140 may be provided to measure a surface temperature of the coil 12 inside the chamber 11. In addition, the temperature sensor 140 may be installed on the measurement target 13 to measure a temperature of the measurement target 13.

The infrared sensor 150 obtains information about the measurement target 13 while moving along a predetermined track inside the chamber 11. The infrared sensor 150 may be installed on the lane 20 and move along the lane. The plurality of infrared sensors 150 may be provided to obtain images of the measurement target 13.

In this case, a passive infrared (PIR) sensor may be used as the infrared sensor 150. The PIR sensor may detect heat energy (infrared rays) emitted from the measurement target 13.

The sensor moving unit 160 provides a driving force such that the infrared sensor 150 installed on the lane 20 moves along the lane 20. The sensor moving unit 160 may include a motor (not shown). The sensor moving unit 160 may detect a position of the infrared sensor 150 by calculating a movement distance of the infrared sensor 150.

The cooling fan 170 may be disposed on the rear surface of the chamber 11 and supply cool air into the interior of the chamber 11 according to fan driving. The cooling fan 170 may measure a surface temperature of the measurement target 13 through the temperature sensor 140 and operate according to the measured surface temperature to adjust a temperature of the measurement target 13.

The pump 180 may be connected to the chamber 11, may operate according to a surface temperature of the coil 12, and supply a coolant to be accommodated in the chamber 11.

The power supply 190 may supply a current with a predetermined magnitude to the coil 12. In response to a control instruction of the processor 110, the power supply 190 may supply a current with a predetermined magnitude, which corresponds to a surface temperature of the coil 12, to the coil 12 according to a surface temperature of the coil 12 measured through the temperature sensor 140.

The processor 110 may include at least one microprocessor and operate according to an algorithm based on data stored in the memory 120.

The processor 110 may apply a control instruction to operate the pump 180 or cooling fan 170 by receiving a surface temperature of the coil 12 and a temperature of the measurement target 13 from the plurality of temperature sensors 140.

The processor 110 may control the pump 180 to supply a coolant such that the coil 12 and the measurement target 13 maintain a constant temperature and may control the power supply 190 to adjust an amount of current supplied to the coil 12. Accordingly, the coil 12 may maintain a constant temperature.

In addition, based on a temperature of the measurement target 13 measured through the temperature sensor 140, the processor 110 may drive the cooling fan 170 and control a rotation speed of the cooling fan 170.

Meanwhile, when the coil 12 and the measurement target 13 maintain a constant temperature, the processor 110 controls the sensor moving unit 160 to allow the infrared sensor 150 to move along the lane 20 to detect infrared rays for the measurement target.

The processor 110 stores infrared data measured by the infrared sensor 150 in the memory 120. The processor 110 may analyze infrared rays and a thermal state of the measurement target 13 by analyzing data input from the infrared sensor 150. The processor 110 may preprocess infrared data to filter unnecessary data and remove noise.

By using the heat generation characteristics of nanomagnetic particles (SPIO) based on infrared data, the processor 110 may determine whether the nanomagnetic particles (SPIO) are located at a designated location and may check a state of biological tissue of the measurement target 13.

In particular, the processor 110 may perform control such that heat kills the lesion by heating a lesion (for example, cancer tissue) located in the measurement target 13 to a high temperature based on the heat generation characteristics of the nanomagnetic particles (SPIO).

In addition, the processor 110 may generate and output a two-dimensional image or a three-dimensional image of the measurement target 13 based on infrared data.

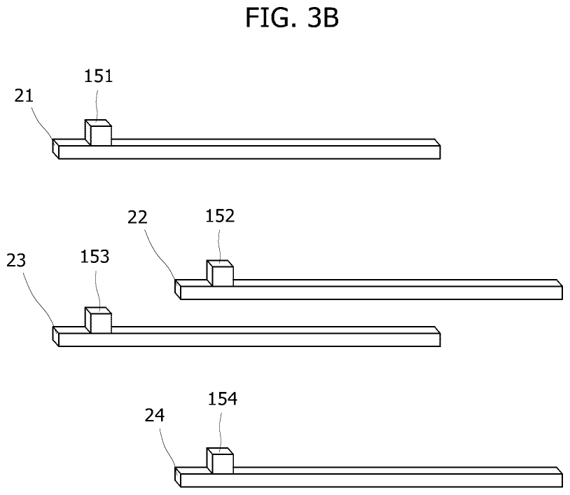

FIGS. 3A and 3B are diagrams illustrating a structure for moving the infrared sensor of the medical imaging device according to one embodiment of the present invention.

As shown in FIGS. 3A and 3B, the infrared sensor 150 is installed on each of first to fourth lanes 21 to 24 to move on the lane 20 to detect infrared rays generated from the measurement target 13. In this case, the infrared sensor 150 may move along the lane 20 at a predetermined speed.

For example, when a first infrared sensor 151 is installed on the first lane 21, a second infrared sensor 152 is installed on the second lane 22, and a third infrared sensor 153 is installed on the third lane 23, the fourth infrared sensor 154 may be installed in the fourth lane 24.

When a magnetic field is generated by the coil 12 while the infrared sensor 150 moves along the lane 20 in the chamber 11, the infrared sensor 150 may detect local heat emission from nanomagnetic particles (SPIO) caused by the generated magnetic field.

In this case, the processor 110 controls the first infrared sensor 151 and the second infrared sensor 152 to move along the first lane 21 and the second lane 22 so as to obtain position information on a YZ plane. The processor 110 may obtain the position information on the YZ plane by analyzing data obtained from the first infrared sensor 151 and the second infrared sensor 152.

The processor 110 controls the first infrared sensor 151 and the third infrared sensor 153 to move on the first lane 21 and the third lane 23 so as to obtain position information on an XZ plane. The processor 110 may obtain the position information on the XZ plane by analyzing data obtained from the first infrared sensor 151 and the second infrared sensor 152.

The processor 110 may obtain three-dimensional position information using the XZ plane and the YX plane. In this case, in consideration of a penetration depth of infrared rays, the processor 110 may obtain information on four planes using the fourth infrared sensor 154 on the fourth lane 24 together.

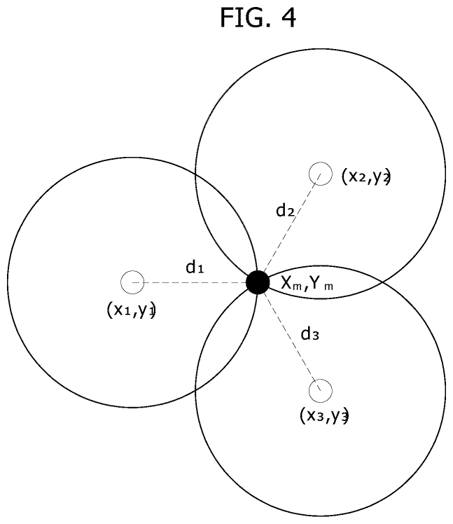

FIG. 4 is an exemplary diagram illustrating a method of designating a heating position of nanomagnetic particles in the medical imaging device according to one embodiment of the present invention.

Referring to FIG. 4, the processor 110 may calculate a heating position of the nanomagnetic particle (SPIO) based on position information of the infrared sensor 150 of FIGS. 3A and 3B.

The processor 110 may calculate a distance between points and a position of a point (Xm, Ym) as in Expression 1 below. In this case, the point (Xm, Ym) is a heating position of the nanomagnetic particles (SPIO).

$$d 1 = \sqrt{{( x 1 - Xm )}^{2} + {( y 1 - Ym )}^{2}} \qquad [ Expression 1 ]$$

$$d 2 = \sqrt{{( x 2 - Xm )}^{2} + {( y 2 - Ym )}^{2}}$$

$$d 3 = \sqrt{{( x 3 - Xm )}^{2} + {( y 3 - Ym )}^{2}}$$

$$\overline{d 1} , \overline{d 2} , \overline{d 3} ⇒ \overline{Xm} , \overline{Ym}$$

Assuming that positions of at least three infrared sensors on least three lanes are a first point (x1, y1), a second point (x2, y2), and a third point (x3, y3), the processor 110 may calculate a position of the point (Xm, Ym), at which the three points meet, based on distances d1, d2, and d3 to respective points.

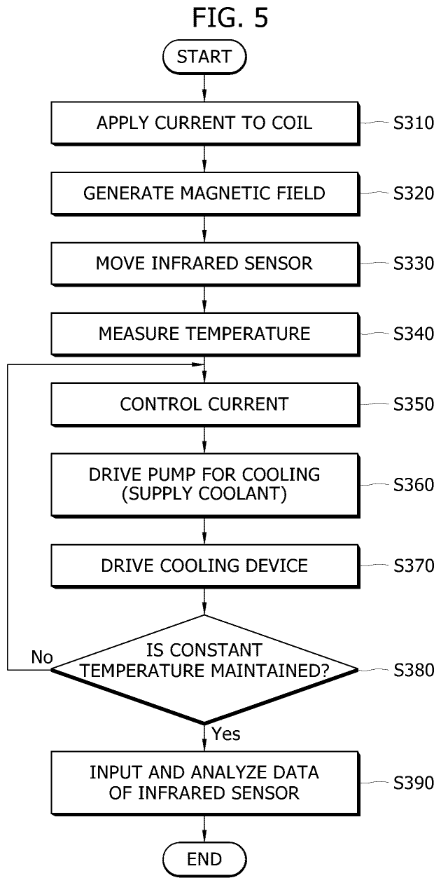

FIG. 5 is a flowchart illustrating an operation method of a medical imaging device according to one embodiment of the present invention.

Referring to FIG. 5, a medical imaging device 100 may apply power to a coil 12 to generate a magnetic field in a chamber 11 in which a measurement target 13 is located and may measure a heating position of nanomagnetic particles (SPIO) for the measurement target 13 using the nanomagnetic particles (SPIO) generated by the generation of the magnetic field.

The processor 110 calculates an amount of current based on a surface temperature of the coil 12 measured by a temperature sensor 140 and controls a power supply (AC power supply) 190 to apply a current with a predetermined magnitude to the coil 12 (S310).

Accordingly, the coil 12 generates a magnetic field in the chamber 11 by the applied current (S320). When a magnetic field is generated in the chamber 11, a field-free line (FFL) may be generated, and the nanomagnetic particles (SPIO) may be generated. In this case, the processor 110 may perform impedance matching such that the coil 12 generates a magnetic field without generating heat due to a current.

Here, the FFL is a line or space in which a magnetic field is zero and is an area on which a magnetic field does not act. In the FFL, plasma particles may freely move without the influence of a magnetic field. Meanwhile, the nanomagnetic particles (SPIO) have unique magnetic properties and are sensitive to a magnetic field.

The nanomagnetic particles (SPIO) may freely move in an FFL, in which a magnetic field is not present, by other forces such as thermal or electric forces. Therefore, when an FFL is formed in a magnetic field, the nanomagnetic particles (SPOI) may move in response to heat of the measurement target 13.

The infrared sensor 150 may move along the lane 20 (S330) to detect infrared rays generated from the measurement target 13 and the nanomagnetic particles (SPIO).

Meanwhile, the processor 110 receives a surface temperature of the coil 12 and a surface temperature of the measurement target 13 through the temperature sensor 140 (S340).

The processor 110 determines an amount of current of the power supply 190 based on the surface temperature of the coil (S350) and may also control a pump 180 to supply a coolant to the chamber 11 (S360).

In addition, the processor 110 may control a cooling fan 170 in response to a temperature of the measurement target 13 (S370).

The processor 110 may control a temperature of the coil 12 by controlling an amount of current of the power supply 190 and driving the pump 180 to inject a coolant. In addition, the processor 110 may control the temperature of the measurement target 13 by driving the cooling fan 170.

The processor 110 determines whether the temperatures of the coil 12 and the measurement target 13 are maintained constant within a set error range (S380).

When the temperature is not maintained constant, the processor 110 repeatedly controls an amount of current, a coolant, and an operation of the cooling fan 170 to maintain the temperatures of the coil 12 and the measurement target 13 constant.

Meanwhile, when the temperatures of the coil 12 and the measurement target 13 are maintained constant, the processor 110 may receive data from the infrared sensor 150, analyze the received data (S390), and thus calculate a heating position of the nanomagnetic particles (SPIO).

The medical imaging device 100 may identify the heating position of the nanomagnetic particles (SPIO) to distinguish the heating position from a position of the heating of a lesion of the measurement target 13 and generate and output a two-dimensional or three-dimensional image of a position of the nanomagnetic particles (SPIO).

Therefore, in an infrared sensor-based nanomagnetic particle medical imaging device using an exothermic reaction of paramagnetic iron oxide particles, and an operation method thereof according to one aspect of the present invention, based on infrared sensors and triangulation, a medical imaging device in which a heat generation function and a magnetic particle imaging (MPI) device are coupled can be formed to effectively analyze lesions.

In addition, in an infrared sensor-based nanomagnetic particle medical imaging device using an exothermic reaction of paramagnetic iron oxide particles, and an operation method thereof according to one aspect of the present invention, it is possible to obtain heat generation information of nanomagnetic particles (SPIO) for lesions at low costs, easily confirm an exothermic reaction in two-dimensional or three-dimensional imaging equipment, and intuitively obtain two-dimensional or three-dimensional information for imaging.

In an infrared sensor-based nanomagnetic particle medical imaging device using an exothermic reaction of paramagnetic iron oxide particles, and an operation method thereof according to one aspect of the present invention, by using a high-temperature generating device as a main device, and based on infrared sensors and triangulation, a medical imaging device in which a heat generation function and a magnetic particle imaging (MPI) device are coupled can be formed to effectively analyze lesions.

In an infrared sensor-based nanomagnetic particle medical imaging device using an exothermic reaction of paramagnetic iron oxide particles, and an operation method thereof according to one aspect of the present invention, it is possible to obtain heat generation information of nanomagnetic particles (SPIO) for lesions and easily confirm an exothermic reaction in two-dimensional or three-dimensional imaging equipment.

In an infrared sensor-based nanomagnetic particle medical imaging device using an exothermic reaction of paramagnetic iron oxide particles, and an operation method thereof according to one aspect of the present invention, it is possible to intuitively obtain two-dimensional or three-dimensional information for imaging.

In an infrared sensor-based nanomagnetic particle medical imaging device using an exothermic reaction of paramagnetic iron oxide particles, and an operation method thereof according to one aspect of the present invention, it is possible to reduce power and costs required to generate and move an FFL.

What is claimed is:

1. An infrared sensor-based nanomagnetic particle medical imaging device comprising:
a coil configured to generate a magnetic field inside a chamber in which a measurement target is accommodated;
an infrared sensor which moves along a lane installed in the chamber to detect infrared rays generated from the measurement target and nanomagnetic particles generated by the magnetic field;
a temperature sensor configured to measure temperatures of the coil and the measurement target; and
a processor configured to control a current applied to the coil to generate the magnetic field in the chamber, analyze data measured through the infrared sensor, and calculate heating positions of the measurement target and the nanomagnetic particles.

2. The infrared sensor-based nanomagnetic particle medical imaging device of claim 1, further comprising a pump configured to inject a coolant into the chamber,
wherein the processor controls the pump and the current according to the temperature of the coil measured by the temperature sensor such that the coil maintains a constant temperature.

3. The infrared sensor-based nanomagnetic particle medical imaging device of claim 1, further comprising a cooling fan configured to supply cold air to the measurement target, wherein the processor controls the cooling fan to be driven in response to the temperature of the measurement target such that the measurement target maintains a constant temperature.

4. The infrared sensor-based nanomagnetic particle medical imaging device of claim 1,
wherein the infrared sensor moves at a predetermine speed along the lane.

5. The infrared sensor-based nanomagnetic particle medical imaging device of claim 1, wherein the lane includes first to fourth lanes, and
the infrared sensor is provided as one or more infrared sensors disposed on each of the first to fourth lanes.

6. The infrared sensor-based nanomagnetic particle medical imaging device of claim 5, wherein the processor obtains information on at least three planes in consideration of a penetration depth of the infrared rays based on data of the infrared sensor measured on the first to fourth lanes.

7. The infrared sensor-based nanomagnetic particle medical imaging device of claim 5, wherein the processor calculates the heating position of the nanomagnetic particles based on position information obtained from at least three infrared sensors among the infrared sensors installed on the first to fourth lanes.

8. The infrared sensor-based nanomagnetic particle medical imaging device of claim 5, wherein the processor generates three-dimensional position information based on position information obtained from at least three infrared sensors among the infrared sensors installed on the first to fourth lanes.

9. The infrared sensor-based nanomagnetic particle medical imaging device of claim 8,
wherein the processor generates a two-dimensional image or a three-dimensional image of the measurement target and the nanomagnetic particles.

10. An operation method of an infrared sensor-based nanomagnetic particle medical imaging device, the operation method comprising:
applying, by a processor, a current to a coil and generating a magnetic field inside a chamber in which a measurement target is accommodated;
receiving, by the processor, data about the measurement target and nanomagnetic particles measured through an infrared sensor; and
analyzing, by the processor, the data measured through the infrared sensor and calculating heating positions of the measurement target and the nanomagnetic particles.

11. The operation method of claim 10, wherein the generating of the magnetic field includes:
receiving, by the processor, a temperature of the coil from a temperature sensor; and
controlling, by the processor, the current applied to the coil or a coolant injection according to the temperature of the coil measured through the temperature sensor.

12. The operation method of claim 11, wherein, in the generating of the magnetic field, the processor controls the coil to maintain a constant temperature.

13. The operation method of claim 10, wherein the generating of the magnetic field includes:
receiving, by the processor, a temperature of the measurement target from a temperature sensor; and
controlling, by the processor, a cooling fan configured to supply cold air to the measurement target.

14. The operation method of claim 10, wherein the receiving of the data includes controlling, by the processor, the infrared sensor to move at a predetermined speed along a lane installed in the chamber.

15. The operation method of claim 14, wherein, in the receiving of the data,
the lane includes first to fourth lanes, and
the infrared sensor is provided as one or more infrared sensors disposed on each of the first to fourth lanes to detect the measurement target and the nanomagnetic particles.

16. The operation method of claim 15, wherein, in the receiving of the data, the processor obtains information on at least three planes in consideration of a penetration depth of infrared rays based on data of the infrared sensor measured on the first to fourth lanes.

17. The operation method of claim 15, wherein, in the calculating of the heating positions of the measurement target and the nanomagnetic particles, the processor calculates the heating position of the nanomagnetic particles based on position information obtained from at least three infrared sensors among the infrared sensors installed on the first to fourth lanes.

18. The operation method of claim 15, wherein, in the calculating of the heating positions of the measurement target and the nanomagnetic particles, the processor generates three-dimensional position information based on position information obtained from at least three infrared sensors among the infrared sensors installed on the first to fourth lanes.

19. The operation method of claim 10, wherein, in the calculating of the heating positions of the measurement target and the nanomagnetic particles, the processor generates a two-dimensional image or a three-dimensional image of the measurement target and the nanomagnetic particles.
